(19) United States
(12) Patent Application Publication
(10) Pub. No.: US 2003/0153512 A1
(43) Pub. Date: Aug. 14, 2003

(54) Curcumin derivatives with improved water solubility compared to curcumin and medicaments containing the same

(76) Inventors: Manfred Hergenhahn, Dossenheim (DE); Barbara Bertram, Heidelberg (DE); Manfred Wiessler, Frankenthal (DE); Bernda L Sorg, Rohrbach (DE)

Correspondence Address: INTELLECTUAL PROPERTY / TECHNOLOGY LAW, PO BOX 14329, RESEARCH TRIANGLE PARK, NC, 27709, US

(21) Appl. No.: 10/312,951

(22) PCT Filed: Jun. 28, 2001
(86) PCT No.: PCT/DE01/02337

(30) Foreign Application Priority Data
Jun. 30, 2000 (DE) 10031955.6

(51) Int. Cl. A61K 31/704; C07H 15/00

(52) U.S. Cl. 514/25; 536/4.1

(57) ABSTRACT

The invention relates to curcumin derivatives with improved water solubility compared to curcumin, which are characterized in that the curcumin part is linked to a monosaccharide, oligosaccharide or polysaccharide, and to medicaments containing these derivatives. The curcumin derivatives according to the invention are particularly suitable to prevent and treat cancer, chronic-inflammatory diseases and diseases associated with a retrovirus infection.

The present invention relates to curcumin derivatives having a water solubility improved as compared to curcumin, which are characterized in that the curcumin part is linked to a saccharide, as well as to medicaments containing these derivatives. The curcumin derivatives according to the invention are particularly suited to prevent and treat cancer, preferably EBV-associated tumors and transplantation-associated lymphoproliferative diseases, chronic-inflammatory diseases and a disease associated with a retrovirus infection.

Curcumin (1,7-bis(4-hydroxy-3-methoxyphenyl)-1,6-heptadiene-3,5-dione; enol form) is the dyestuff of curcuma plants, e.g. of Curcuma xanthoriza and Curcuma domestica, and in animal experiments has proved to be a highly effective chemopreparative substance within the meaning of a tumor-inhibiting effect thus far, with virtually no toxic effects being observable. Curcumin also has a strongly anti-inflammatory effect. Finally, curcumin analogues showed a good inhibitory effect as regards the integrase of HIV in cell cultures (Mazumder et al., J. Med. Chem. 40, pp. 3057-3063). They can thus prevent the integration of HIV-DNA following reverse transcription into the host genome. This integration is the precondition for an efficient replication of these viruses in the host cells, which in the case of HIV is associated with the disease prognosis. An inhibition of integrase by means of curcumin and/or curcumin analogues can thus be regarded as an important therapeutic measure serving for controlling an HIV infection. As regards curcumin as such, similarly good results as obtained in animal models thus far could not be achieved in humans in clinical phase I and phase II studies, it being assumable that this is based on the fact that the active substance is not available in sufficient concentration at the site of action. Data obtained from a clinical phase I study in Taiwan show that with an administration of 4 g/day curcumin serum concentrations of only 0.41 μM, with 6 g/day of 0.57 μM and with 8 g/day of 1.75 μM were achieved, i.e. only a minor curcumin portion reaches the circulation and the major part is removed without being made use of. In this study, curcumin was administered in the form of sucking tablets of 100 mg each or 1 g active substance in a morning dose up to 8 g over a period of several months. It is probable that with the measured minor serum concentrations no effect could be achieved, since the different chemopreventive effects of curcumin in cell cultures, which are described in the literature, only occurred at concentrations of at least 10 μM in the medium. Finally, the procedure of the Taiwan study also has another serious drawback. In order to be able to achieve a fairly good uptake of curcumin in the mouth and throat regions at all, the patients should melt the curcumin tablets in their mouths. Since it took at least 15 minutes until each single tablet of the tablet type used in the Taiwan study was dissolved, the patients would have to melt one tablet each in their mouths over a relatively long time of the day for several months. It appears at least doubtful whether the patients would have the necessary compliance for this.

It is thus the object of the present invention to provide curcumin, curcumin derivatives or curcumin analogues in such a form that relatively high plasma concentrations and thus therapeutically active concentrations can be achieved upon administration. This should also serve for reducing the therapeutically necessary daily dose, which should add to the patient's readiness to complete the therapy.

This technical problem is solved by the embodiments characterized in the claims.

It was assumed in the present invention that the minor activity of orally supplied curcumin in humans is a result of the poor solubility of curcumin which then results in poor concentrations in the serum. Therefore, curcumin derivatives were developed which have an increased solubility because they are linked to saccharide residues. These curcumin derivatives can thus be supplied e.g. as prodrugs by oral administration, injection or infusion and should enable plasma levels to be permitted with which a therapeutic effect can be achieved. This procedure is advantageous also because the diketone structure is not destroyed in this case; this structure is obviously necessary for the curcumin effect. The curcumin derivatized according to the invention does not only have an increased solubility but it can also be assumed that it is taken up in a better way by the cells because specific transport systems are available on the cell surface for this purpose (Veyl et al., PNAS 95, pp. 2914-2919 (1998)). Therefore, compounds according to the invention accumulate preferably in cells, organs and tissues which have glucose transporters and/or related transporters. The conjugates should accumulate in particular in the liver, kidneys, heart, thymus, thyroid gland, intestine and brain as well as in all kinds of tumors. A well-calculated targeting of certain tumor cells can also be achieved by the saccharide structures. For example, the curcumin derivatives should, above all, be better taken up by (pre)malignant cells with expression of the glucose cotransporter SAAT1 or similar cotransporters.

Hence the present invention relates to a curcumin derivative having a water solubility improved as compared to curcumin, characterized in that the curcumin part is linked to a saccharide. This is preferably effected via a glycosidic bond.

The expression “curcumin” used herein relates to both curcumin and curcumin-like compounds having comparable activity and analogues thereof which have a substantially equal solubility. They comprise e.g. dicaffeoylmethane, rosemarinic acid, acrylamides of curcumin as well as the compounds NSC 158393 and NSC 117027 (Mazumder et al., J. Med. Chem. 1996, 39: 2472-2481).

Curcumin and its analogues or derivatives can be linked to the saccharide by means of methods known to a person skilled in the art, e.g. via the Koenigs-Knorr reaction or via the known imidate method. Another suitable method is a method analogous to Artico et al. (J. Med. Chem. 41, pp. 2984, 3960, (1998)) in which according to the invention the curcumin derivatives are obtained by condensation of equimolar mixtures of e.g. 4-hydroxy-3-methoxy-benzaldehyde-4-saccharide and 4-hydroxy-3methoxy-benzaldehyde with acetylacetone with boric acid catalysis and subsequent chromatographic separation of the desired monosaccharides or disaccharides. An alternative synthesis pathway is the production of saccharide derivatives by a “reverse cleavage” in which the reaction equilibrium of the 3-glycosidase cleavage is moved to the left by suitable measures (Menzler et al., 1997, Biotechnology Letters 11 (2), pp. 269-272).

The biological effectiveness of the resulting curcumin derivatives can be checked by means of the known biological properties, e.g. it is possible to check the curcumin-like antioxidative effect or the inhibition of the transcription of numerous viral and celullar genes controlled by promoters containing AP-1 and NF-kappaB sites.

The expression “water solubility improved as compared to curcumin” which is used herein refers to such a water solubility that a sufficiently high concentration of the active substance can be obtained in the serum with oral administration, injection or continuous supply, e.g. for a desired cancer prevention (under certain circumstances also a cancer chemotherapy), anti-inflammatory or virus-inhibitory effect. The serum concentration to be achieved ranges preferably from at least 5-10 μM. A range of 10-30 μM is particularly preferred.

The term “saccharide” comprises saccharides of any kind, in particular, monosaccharides, disaccharides, oligosaccharides or polysaccharides (e.g. mono-, di-, tri-, multi-antennary as well as dendritic saccharides) in all stereoisomeric and enantiomeric forms. These may be pentoses or hexoses. In particular glucose, more particularly α- and β-D-glucose, fructose, galactose, mannose, arabinose, xylose, fucose, rhamnose, digitoxose and derivatives thereof are preferred as monosaccharides. In particular saccharose, maltose, lactose or gentobiose, either 1,4- or 1,6-linked, as well as derivatives thereof are appropriate disaccharides. Inositols and derivatives thereof, in particular cis-inositol, epi-inositol, allo-inositol, myo-inositol, muco-inositol, chiro-inositol, neo-inositol, scyllo-inositol, pinpollitol, streptamine, quercitol, chinic acid, shikimic acid, conduritol A and B, validatol and quebrachitol, e.g. from galactinols, from both vegetable sources, such as sugar beets, and milk products or compounds obtained by enzymatic enantiomer separation are also considered saccharides herein. Furthermore, saccharides usable according to the invention are glycoconjugates. These may be conjugates of e.g. saccharides with peptides, lipids, acids (→esters), alkyl residues (→ethers), heterocycles or other carbohydrates. An example of glycoconjugates is Z1-Z10, a mixture of 10 glycoconjugates. The Z1-Z10 compounds are naturally occurring glycopeptides, glycoproteins and lipopolysaccharides. Derivatives of said saccharides are e.g. saccharides protected with protecting groups, such as benzyl groups, protected saccharides and/or saccharides modified with functional groups, such as amino groups, phosphate groups or halide groups. According to the invention saccharides are also understood to mean whole saccharide libraries, as described in German patent application DE 196 42 751.7, for example. The above saccharides may occur in nature or be produced synthetically. A conjugate according to the invention preferably has only one saccharide, but a number of 2, 3, 4, 5 and 6 saccharide components is also conceivable. In this case, the saccharides may be equal or differ from one another.

In a preferred embodiment of the conjugates according to the invention, a saccharide is linked to the curcumin component via a linker. Appropriate linkers are in particular short-chain diols from 1,2-diol (e.g. ethylene glycol) to 1,6-hexanediol. Ether bridges and dicarboxylic linkers can also be used.

The derivatization is preferably carried out such that the active substance (curcumin, curcumin-like compounds or analogues) is again released in the target cell by spontaneous or enzymatically supported hydrolysis. This can be done e.g. by extracellular or intracellular B-glucosidase(s) which show a broad spectrum of activities as compared to glycoside derivatives and are available in human liver and the small intestine at the relatively highest concentrations as compared to other human organs. Thus, in a preferred embodiment of the present invention the curcumin derivatives according to the invention are characterized in that the linkage with the monosaccharide, oligosaccharide or polysaccharide is effected such that by spontaneous, acid-catalyzed or enzyme-catalyzed hydrolysis due to the acid-labile sugar bond at the phenolic OH group the active substance curcumin is restored in the target cell, it being possible to check the cleavability in vitro beforehand. Curcumin derivatives in which the linkage is an O-glycosidic bond are particularly preferred. The binding takes place particularly at the 1 or 4 position of the saccharide, the 1 position being preferred for better cleavability.

In the most preferred embodiment, the curcumin derivative according to the invention is the curcumin-4-monoglycoside or curcumin-4,4′-diglycoside and/or the corresponding galactoside.

Finally, the present invention relates to a medicament which contains a curcumin derivative according to the invention, optionally in combination with a pharmaceutically acceptable carrier. Appropriate carriers and the formulation of such medicaments are known to a person skilled in the art. Appropriate carriers are e.g. phosphate-buffered common salt solutions, water, emulsions, e.g. oil/water-emulsions, wetting agents, sterile solutions, etc. The medicament according to the invention may be available in the form of an injection solution, tablet, ointment, suspension, emulsion, a suppository, etc. It may also be administered in the form of depots (microcapsules, zinc salts, liposomes, etc.). The kind of administration of the medicament depends inter alia on the form in which the active substance is available, it may be given orally or parenterally. The methods of parenteral administration comprise the topical, intra-arterial, intra-tumoral (e.g. directly to a carcinoma), intramuscular, intramedullary, intrathekal, intraventricular, intravenous, intraperitoneal, transdermal or transmucosal (nasal, vaginal, rectal, sublingual) administration. The administration can also be made by microinjection. The appropriate dosage is determined by the attending physician and depends on various factors, e.g. on the patient's age, sex and weight, the kind and stage of the disease, the kind of administration, etc.

The curcumin derivatives according to the invention can be administered together with glycosidases (e.g. cerebrosidase), which renders the release of curcumin independent of glycosidases already available in the body. Both components can be packed in liposomes and administered.

Since the tumor-inhibiting effect of curcumin has already been shown by way of animal experiment but was markedly less in humans due to the minor solubility of curcumin, it can be assumed that due to the markedly improved uptake and the resulting substantially higher plasma levels a cancer-inhibitory effect can be achieved with the curcumin derivatives according to the invention. Thus, the present invention relates to the use of the curcumin derivative according to the invention for the prevention or treatment of cancer. It was also possible to show an inhibition of the Epstein-Barr virus reactivation in B-lymphoid cells. On account of the strongly improved solubility of the curcumin derivatives according to the invention it can be assumed that the curcumin derivatives according to the invention are also suited to the prevention or treatment of EBV-associated tumors, e.g. the nasopharyngeal carcinoma; EBV-containing Hodgkin's lymphomas and EBV-containing non-Hodgkin's lymphomas, EBV-containing T-cell lymphomas, EBV-containing gastric cancers, EBV-associated HCV hepatitis, EBV-associated tumors of the female breast and transplantation-associated lymphoproliferative diseases (PTLD). Thus, the present invention also relates to the use of the curcumin derivatives according to the invention for preventing or treating EBV-associated tumors and transplantation-associated lymphoproliferative diseases. The same also applies to other viruses, such as hepatitis B viruses and human papilloma viruses, in which important genes are controlled via protein kinase C, NF-kappa B, Jun kinases and AP1 sites, as well as the diseases and tumors associated with these diseases.

The present invention also relates to the use of the curcumin derivatives according to the invention for treating chronic-inflammatory diseases. What matters here is the antioxidative effect of curcumin and/or curcumin derivatives as compared to reactive oxygen species from inflammatory cells.

Since it has already been shown that curcumin analogs in cell cultures have an inhibitory effect on the integrase of HIV, for example, it can be assumed that due to the improved properties an effective antiviral therapy in humans can be achieved with the curcumin derivatives according to the invention. Thus, the present invention finally relates to the use of the curcumin derivatives according to the invention for treating diseases accompanied by a retrovirus infection, preferably by a HIV infection.

### DESCRIPTION OF THE FIGURES

FIG. 1: Diagram of the synthesis of curcumin monoglycoside

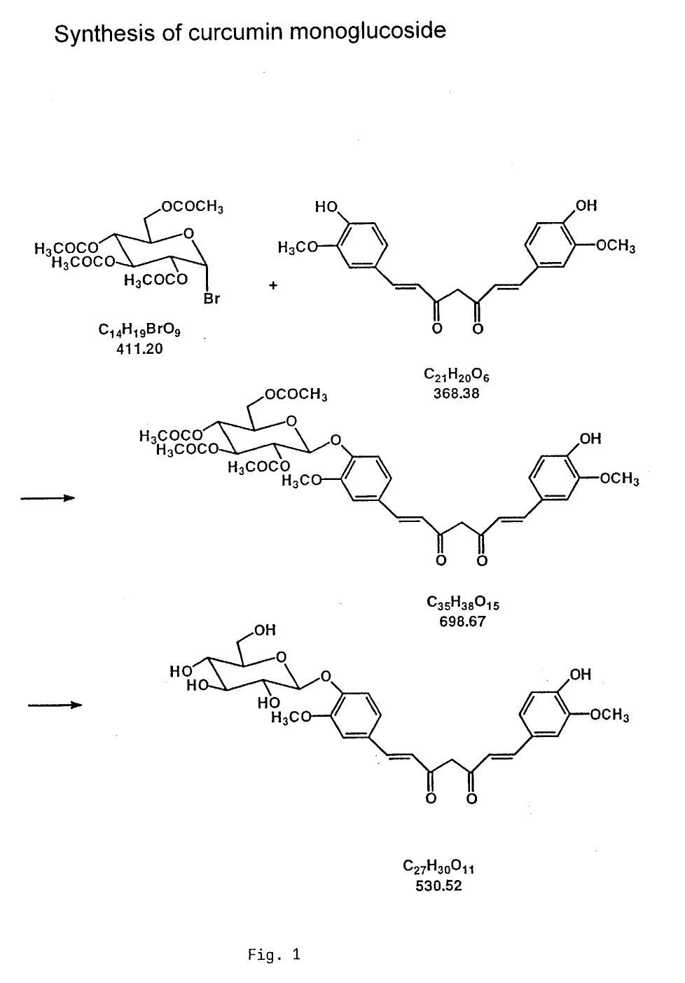

FIG. 2: Diagram of the synthesis of curcumin diglyocside

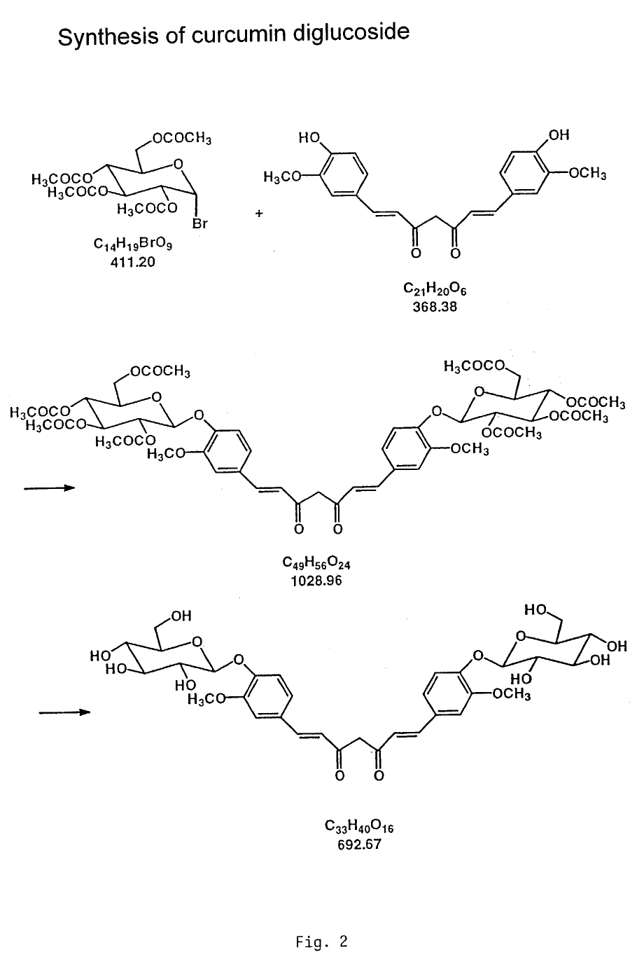

FIG. 3: Inhibition of the “oxygen burst” of granulocytes

FIG. 4: Inhibition of EBV induction in Raji-DR-Luc cells

The following examples explain the invention.

### EXAMPLE 1

### Production of 8-(2″, 3″, 4″, 6″-tetra-O-acetyl-β-D-glucopyranosyl)curcumin (1) and 8-(2″, 3″, 4″, 6″-tetra-O-acetyl-β-D-glucopyranosyl)-8′-(2″′, 3″′, 4″′, 61″′-tetra-O-acetyl-β-D-glucopyranosyl)curcumin (2)

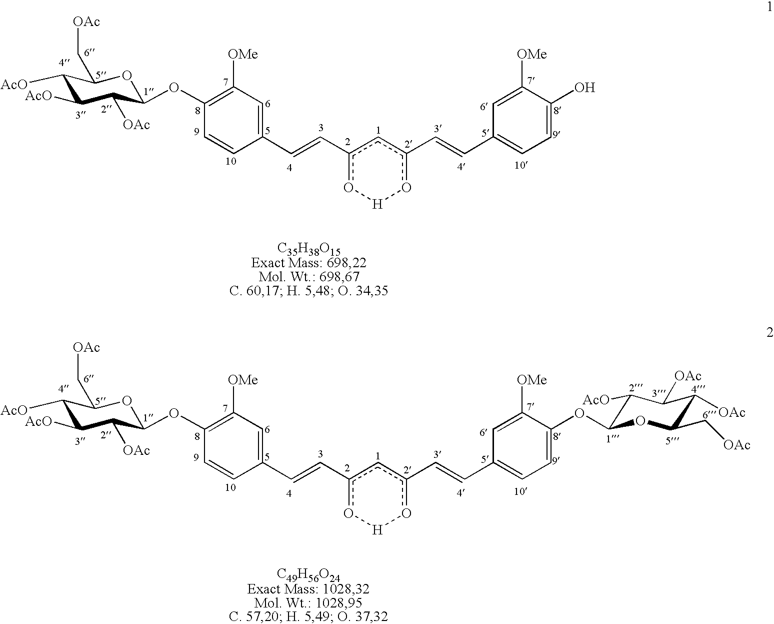

A mixture of 0.87 g (3.2 mmol) benzyltriethylammonium-bromide, 8.25 ml 1.25 M caustic soda solution, 16 ml dichloromethane, 1.64 g (4 mmol) α-D-acetobromoglucose and 2.9 g (8 mmol) curcumin were stirred intensively at 60° for 12 h. After cooling down to room temperature, the organic phase was separated, shaken out twice with saturated aqueous sodium chloride solution and washed twice with water. Thereafter, the organic phase was dried on sodium sulfate, filtered off and concentrated in vacuo. Purification was made by means of column chromatography (silica gel, petroleum ether/acetic acid ethyl ether 1.1:1 for elution of the monoglucoside, petroleum ether/acetic acid ethyl ester 5:8 for elution of the diglucoside).

Yield: 0.395 g (14% of the theoretical): monoglucoside 0.461 g (11% of the theoretical): diglucoside 2

Analytical Data of 1:

NMR: δ_{H} (250 MHz, CDCl₃, 30° C., TMS): 2.04 (s, 6H, COCH₃), 2.08 (s, 6H, COCH₃), 3.80 (ddd, 1H, J_{4″,5″} 9.8, J_{5″,6a″} 2.5, J_{5″,6b″} 4.9, 5″-H), 3.86 (s, 3H, OCH₃), 3.94 (s, 3H, OCH₃), 4.18 (dd, 1H, J_{5″,6a″} 2.5, J_{6a″,6b″} 12.3, 6a″-H), 4.29 (dd, 1H, J_{5″,6b″} 4.9, J_{6a″,6b″} 12.3, 6b″-H), 5.01-5.31 (m, 4H, 1″-H, 2″-H, 3″-H, 4″-H), 5.81 (s, 1H, 1-H), 5.92 (bs, 1H, OH), 6.48 (d, 1H, J_{3,4} oder J_{3′,4′} 15.9, CHCHCO), 6.51 (d, 1H, J_{3,4} oder J_{3′,4′} 15.9, CHCHCO), 6.91-7.14 (m, 6H, 6-H, 9-H, 10-H, 6′-H, 9′-H, 10′-H), 7.57 (d, 1H, J_{3,4} oder J_{3″,4″} 15.9, CHCHCO), 7.60 (d, 1H, J₃,₄ oder J₃₋₄-15.9, CHCHCO)

δ_{C} (63 MHz, CDCl₃, 30° C.): 20.57, 20.60, 20.67 (COCH₃), 55.96, 56,12 (7-OCH₃, 7′-OCH₃) 61.93 (6″-C), 68.38, 71.17, 72.12, 72.54 (2″-C, 3″-C, 4″-C, 5″-C), 100.32, 101.37, 109.71, 111.68, 114.87, 119.64, 121.51, 121.75, 122.96, 123.50 (1-C, 1″-C, 3-C, 3′-C, 6-C, 6′-C, 9-C, 9′-C, 10-C, 10′-C), 127.58, 131.69 (5-C, 5′-C), 139.52, 140.97 (4-C, 4′-C), 146.83, 147.63, 148.00, 150.79 (7-C, 7′-C, 8-C, 8′-C), 169.29, 169.38, 170.23, 170.54 (COCH₃), 182.22, 184.13 (2-C, 2′-C)

Mass spectroscopy: pos. ESI-MS (MeOH/CHCl₃ 2:1): m/z (%): 699.1 [M+H]⁺ (16), 721.1 [M+Na]⁺ (100), 1419.6 [2M+Na]⁺ neg. ESI-MS (MeOH/CHCl₃ 2:1): m/z (%): 697.1 [M−H]⁻ (100), 733.0 [M+Cl]⁻ (8)

TLC: Silica gel, petroleum ether/acetic acid ethyl ester (1.1:1) R_{f}=0.20

Analytical Data of 2:

NMR: δ_{H} (250 MHz, CDCl₃, 30° C., TMS): 2.04 (s, 12H, COCH₃), 2.08 (s, 12H, COCH₃), 3.81 (ddd, 2H, J_{4″,5″} 9.8, J_{5″,6a″} 2.5, J_{5″,6b″} 5.0, J_{4′″,5′″} 9.8, J_{5′″,6a′″} 2.5, J_{5′″,6b′″} 5.0, 5″-H, 5′″-H), 3.87 (s, 6H, 7-OCH₃, 7′-OCH₃), 4.18 (dd, 2H, J_{5″,6a″} 2.5, J_{5″,6b″} 5.0, J_{6a″,6b″} 12.2, J_{5′″,6a′″} 2.5, J_{5″″,6b′″} 5.0, J₆a′″,6b′″ 12.2, 6a″-H, 6a′″-H), 4.29 (dd, 2H, J_{5′,6b′} 5.0, J_{6a″,6b″} 12.2, J_{5′″,6b′″} 5.0, J_{6a′″,6b′″} 12.2, 6b ′-H, 6b ′″-H), 5.01-5.31 (m, 8H, 1′-H, 1′″-H, 2″-H, 2′″-H, 3″-H, 3′″-H, 4″-H, 4′″-H), 5.84 (s, 1H, 1-H), 6.52 (d, 2H, J_{3,3′} 16.0, 3-H, 3′-H), 7.08-7.14 (m, 6H, 6-H, 9-H, 10-H, 6′-H, 9′-H, 10′-H), 7.59 (d, 2H J_{4,4′} 16.0, 4-H, 4′-H)

δ_{C} (63 MHz, CDCl₃, 30° C.): 20.54, 20.58, 20.66 (COCH₃), 56.12 (7-OCH₃, 7′-OCH₃), 61,90 (6″-C, 6′″-C), 68.35,71.15, 72.10, 72.50 (2″-C, 2′″-C, 3″-C, 3′″-C, 4″-C, 4′″-C, 5″-C, 5′″-C), 100.27, 101.56 (1-C, 1″-C, 1′″-C), 111.70, 119.61, 121.59, 123.45 (3-C, 3′-C, 6-C, 6′-C, 9-C, 9′-C, 10-C, 10′-C), 131.55 (5-C, 5′-C), 139.96 (4-C, 4′-C), 147.72, 150.78 (7-C, 7-C, 8-C, 8′-C), 169.26, 169.36, 170.21, 170.51 (COCH₃), 183.10 (2C, 2′-C)

Mass spectroscopy: pos. ESI-MS (MeOH/CHCl₃ 2:1): m/z (%): 1029.3 [M+H]⁺ (90), 1051.3 [M+Na]⁺ (12), 699.1 [Monoglucosid 1+H]⁺ (80), 2057.7 [2M+H]⁺

TLC: Silica gel, petroleum ether/acetic acid ethyl ester (5:8) R_{f}=0.22

### EXAMPLE 2

### Production of 8-(β-D-glucopyranosyl)curcumin (3)

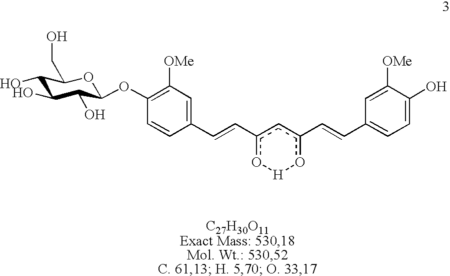

0.375 g (0.51 mmol) acetyl-protected curcumin glucoside 1 was taken up in 40 ml methanol, stirred, mixed with 10 ml of a 0.1 M sodium methanolate solution and further stirred at room temperature for 3 h. Thereafter, the mixture was neutralized using the ion-exchange resin Amberlite H⁺ 50 WX 2, filtered off and concentrated in vacuo. The reaction mixture was separated by means of column chromatography (silica gel, dichloromethane/methanol 9:1)

Yield: 0.151 g (56% of the theoretical)

TLC: Silica gel, dichloromethane/methanol (9:1) R_{f}=0.22

Analytical Data of 3:

NMR: δ_{H} (250 MHz, CD₃OD, 30° C., TMS): 3.42-3.89 (m, 6H, 2″-H, 3″-H, 4″-H, 5″-H, 6a″-H, 6b″-H), 3.89 (s, 6H, 7-OCH₃, 7′-OCH₃), 4.96 (d, 1H, J_{1″,2″} 7.4, 1″-H), 5.96 (s, 1H, 1-H), 6.60 (d, 1H, J_{3,4} oder J_{3′,4′} 15.8, CHCHCO), 6,65 (d, 1H, J_{3,4} oder J_{3″,4″} 15.8, CHCHCO), 6.79-7.23 (m, 6-H, 6′-H, 9-H, 9′-H, 10-H, 10′-H), 7.54 (d, 1H, J_{3,4} oder J_{3′,4′} 15.8, CCHCHCO), 7.56 (d, 1H, J_{3,4} oder J_{3′,4′} 15,8, CHCHCO)

δ_{C} (63 MHz, CD₃OD, 30° C.): 56.48,56.79 (7-OCH₃,7-OCH₃),62.52 (6″-C), 71.33,74.85, 77.88,78.31 (2″-C, 3″-C, 4″-C, 5″-C) 102.29 (1″-C) 111.84, 112.51(6-C, 6′-C), 116.61, 117.52 (9-C, 9′-C), 122.33, 123.44, 123.87, 124.21(3-C, 3′-C, 10-C, 10′-C), 128.53, 131.42 (5-C, 5′-C), 141.10, 142.51(4-C, 4′-C), 149.43, 149.88, 150.57, 151.06 (7-C, 7′-C, 8-C, 8′-C), 183.71, 185.65 (2-C, 2′-C)

Mass spectroscopy: pos. ESI-MS (MeOH): m/z (%):530.6 [M+H]⁺ (4), 552.9 [M+Na]⁺ (100) neg. ESI-MS (MeOH): m/z (%):528.9 [M−H]⁻ (100), 564,9 [M+C]⁻ (4)

m/z (pos. FAB, nitrobenzyl alcohol): found 531.1896 [M+H]⁺, calculated for C₂₇H₃₁O₁₁ 530.1857

### EXAMPLE 3

### Production of 8,8′-bis-(B-D-glucopyranosyl)-curcumin (4)

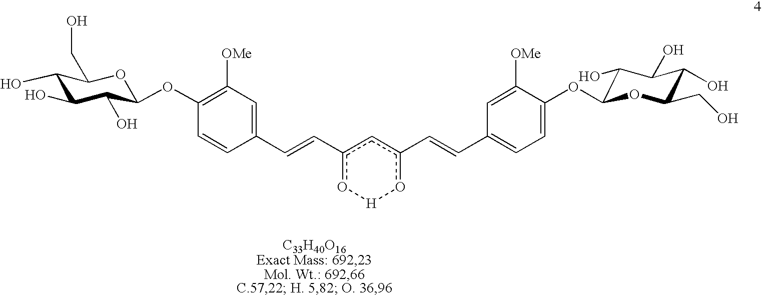

0.381 g (0.37 mmol) acetyl-protected curcumin glucoside 2 was taken up in 40 ml methanol, stirred, admixed with 10 ml of a 0.1 M sodium methanolate solution and further stirred at room temperature for 3 h. Thereafter, the mixture was neutralized with the ion-exchange resin Amberlite H⁺ 50-wx 2, filtered off and concentrated in vacuo. The reaction mixture was separated by means of column chromatography (silica gel, dichloromethane/methanol 4:1).

Yield: 0.071 g (28% of the theoretical)

Analytical Data of 4:

Mass spectroscopy: pos. ESI-MS (MeOH/H₂O 2:1): m/z (%):693.1 [M+H]⁺ (4), 715.2 [M+Na]⁺ (72), 1408.2 [2M+Na]⁺ (8) neg. EST-MS (MeOH/H₂O 2:1): m/z (%): 691.1 [M−H]⁻ (88), 727.1 [M+Cl]⁻ (92)

HR-FAB: m/z (pos. FAB, glycerin): found 693.2408 [M+H]⁺, calculated for C₃₃H₄₁O₁₆ 692.2382

TLC: Silica gel, dichloromethane/methanol (4:1) R_{f}=0.20

### EXAMPLE 4

### Water Solubility of Curcumin, Curcumin Monoglucoside and Diglucoside

Curcumin and its two derivatives (1) and (2) can be separated by means of a HPLC system and quantified.

HPLC conditions: column lichrospher-100-RP18-5 μ, 125×4 mm Running agent/gradient/flow Acetonitrile/acetic acid 0.2 and/or 2%, 1 ml/min Detection: UV 420 nm

The relevant straight calibration line was respectively determined with the individual compounds. For determining the water solubility of curcumin and its two derivatives, saturated solutions were prepared at room temperature in argon-saturated water. The content of 20 μl each of the saturated solutions was determined by means of HPLC.

The following values were initially obtained for the solubility:

Curcumin: 4+/−0.3 mg/liter

Curcumin monoglucoside 13.2+/−0.9

Curcumin diglucoside>10700

This leads to an increased water solubility of the two derivatives, above all of the diglucoside with which a saturated solution could not be obtained at all using the amount employed.

### Example 5

### Uptake of Curcumin and Its Derivatives (1) and (2) in Raji Cells

These studies were carried out by means of fluorescence microscopy, since curcumin and its two derivatives show fluorescence.

It was known from preliminary tests that curcumin shows maximum uptake in Raji cells after 2-3 hours. Raji cells were thus incubated in each case for 3 hours with 15 μM curcumin or derivatives each.

Aliquots of the cells in medium were placed by pipetting onto poly-L-lysin-coated slides (to adhere the cells); the cells were covered with a cover glass after 30 minutes and stimulated by an appropriate filter using U.V. light. The fluorescence of representative cells was measured by means of image analysis; the very bright fluorescence of cell-associated curcumin showed that above all this compound is taken up by the cells, however, the cells treated with curcumin monoglucoside also showed fluorescence, which was reduced by a factor of 10-100, while the cells treated with curcumin diglucoside were just visible.

This points to an uptake of either the unchanged compounds or their hydrolysis product curcumin in B-lymphoid cells within the meaning of this patent application.

### Example 6

### Studies Regarding the Antioxidative Effect of Curcumin and Its Derivatives (1) and (2)

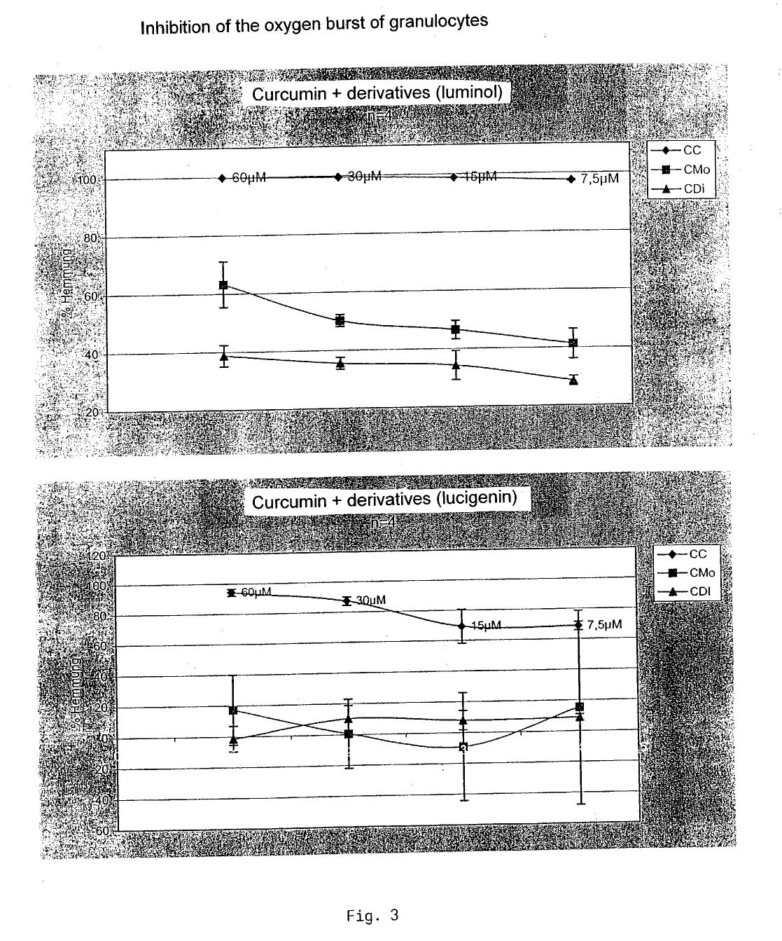

These studies were carried out by means of the inhibition of the “oxygen burst” of granulocytes (PMN) from human blood according to Hergenhahn et al. (1991) J. Cancer Res. Clin. Oncol. 117, 385-395, and Bouvier, Hergenhahn et al. (1993) Carcinogenesis 14, 1573-8. The granulocyte fraction from heparinized blood is enriched by agglutination of the erythrocytes with 2.5% dextran in PBS (30 min. at room temperature), freed from residual erythrocytes by lysis and used after washing with PBS in the test. In a total volume of 1 ml, 100,000 granulocytes each are admixed with luminol and/or lucigenin and heated to 37° C. in the chemiluminescence (CL) measuring device Biolumat LB 953 (EG&G Berthold, Wildbad); the cells are then stimulated with TPA at a final concentration of 100 nM. The CL curves are tracked for one hour; the integral underneath the curve is used as a standard for chemiluminescence. The results are shown in FIG. 3.

When inhibitory substances are used, a concentration-dependent inhibitory effect results which manifests itself as a late rise, reduced amplitude of the maximum and partially as an early drop of the peak.

In this way, a strong antioxidative effect was determined in the Series curcumin >>curcumin monoglucoside curcumin diglucoside.

It can be derived therefrom that curcumin and its derivatives also have antiinflammatory activity, since depending on the concentration they also suppress essential “reactive oxygen species” of inflammatory cells such as granulocytes and macrophages.

### Example 7

### Studies Regarding the Inhibitory Effect on the Reactivation of the Epstein-Barr Virus in Raji Cells

The current studies were carried out with Raji-DR-LUC cells.

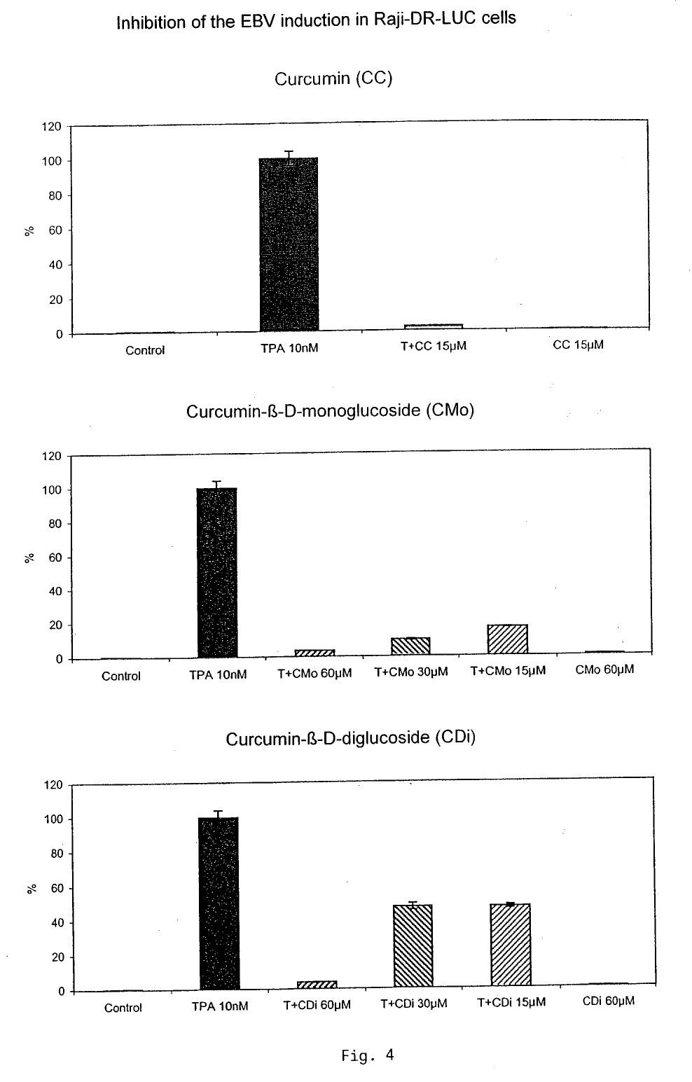

For this purpose, 50,000 Raji-DR cells were treated with 10 nM TPA minus/plus inhibitor (various concentrations) in a volume of 100 μl in a CO2 incubator for 72 hours. Following washing with PBS, the cells are lyzed; the luciferase activity is determined in luciferase buffer (20 mM tricins, 8 mM MgSO₄, 0.1 mM EDTA, 30 mM DTE, pH 7.8) with luciferin/CoA ATP as a substrate in an always equal amount of cell lyzate. The induced luciferase activity per μg protein is determined in comparison with a calibration curve having an authentic luciferase. Inhibiting agents of EBV induction result in an induction of luciferase reduced as compared with the TPA control. The result is shown in FIG. 4.

The strong EBV inhibitory effect determined in this way decreased in the series curcumin>curcumin monoglucoside>curcumin diglucoside.

Curcumin per se and curcumin released from derivatives inhibit the transactivation of important genes, e.g. of the Epstein-Barr virus, the hepatitis B virus, some human papilioma viruses (e.g. HPV 16,18) and further viruses pathogenic for man, via what is called AP1 sequences, via NF-kappa B (family) sequences and via certain signal transduction paths, e.g. PKC-dependent, JNK-dependent paths; however, they presumably have further cellular points of attack as can be derived from their antioxidative effect. From this it can be concluded that they can inhibit the reactivation of the Epstein-Barr virus and inflammatory processes when they reach sufficiently high concentrations in human tissues. Under the same conditions, the synthesis and effect of important AP1-regulated proteins of hepatitis B virus and some human papilloma viruses can also be inhibited under the same conditions.

1. Curcumin derivatives having a water solubility improved as compared to curcumin, characterized in that the curcumin part is linked to one or several saccharide portions, the saccharide being no glucuronic acid.

2. The curcumin derivatives according to claim 1, characterized in that the linkage takes place by means of a monosaccharide, oligosaccharide or polysaccharide.

3. The curcumin derivatives according to claim 2, wherein the linkage takes place by means of the monosaccharide, oligosaccharide or polysaccharide such that the active substance curcumin is released in the target cell by spontaneous or enzymatic hydrolysis.

4. The curcumin derivatives according to claim 1, 2 or 3, wherein the linkage is a glycosidic bond or effected by means of a linker.

5. The curcumin derivatives according to any of claims 2 to 4, wherein the monosaccharide is glucose.

6. The curcumin derivatives according to claim 5, which are curcumin-4-monoglycoside or curcumin-4,4′-diglycoside.

7. A medicament containing curcumin derivatives having a water solubility improved as compared to curcumin, wherein the curcumin part is linked to one or several saccharide portions.

8. Use of a curcumin derivative having a water solubility improved as compared to curcumin, wherein the curcumin part is linked to one or several saccharide portions, for the prevention or treatment of cancer.

9. Use of a curcumin derivative having a water solubility improved as compared to curcumin, wherein the curcumin part is linked to one or several saccharide portions, for the prevention or treatment of EBV-, HBV- or HPV-associated tumors or transplantation-associated lymphoproliferative diseases.

10. Use of a curcumin derivative having a water solubility improved as compared to curcumin, wherein the curcumin part is linked to one or several saccharide portions, for treating chronic-inflammatory diseases.

11. Use of a curcumin derivative having a water solubility improved as compared to curcumin, wherein the curcumin part is linked to one or several saccharide portions, for treating diseases associated with a retrovirus infection.

12. Use according to claim 11, wherein the retrovirus infection is an HIV infection.
